(19) United States
(12) Patent Application Publication
(10) Pub. No.: US 2019/0090975 A1
(43) Pub. Date: Mar. 28, 2019

(54) SURGICAL ILLUMINATION TOOL WITH PROBE

(71) Applicant: Edgardo H. HERNANDEZ, Tempe, AZ (US)

(72) Inventor: Edgardo H. HERNANDEZ, Tempe, AZ (US)

(21) Appl. No.: 16/102,697

(22) Filed: Aug. 13, 2018

Related U.S. Application Data
(60) Provisional application No. 62/544,778, filed on Aug. 12, 2017.

(51) Int. Cl. A61B 90/35; A61B 18/14

(52) U.S. Cl. CPC A61B 90/35; A61B 2090/309; A61B 18/1402

(57) ABSTRACT

A hand-held surgical illumination tool includes an illumination source at its proximal end that projects light into a surgical procedure operating region in which one or more surgical devices interact with a tissue of a patient. In certain embodiments, the hand-held surgical illumination tool includes the one or more surgical devices.

### CROSS-REFERENCE TO RELATED APPLICATION

This application claims priority to U.S. Provisional Application Ser. No. 62/544,778 filed on Aug. 12, 2017, titled SURGICAL ILLUMINATION TOOL WITH PROBE by inventors Edgardo H. HERNANDEZ, the entire disclosure of which is hereby incorporated herein by reference.

### FIELD

Implementations generally relate to a medical tool and medical procedure using that medical tool, and more particularly, to a surgical illumination tool, and most particularly to a cauterizing hand-held surgical illumination tool.

### BACKGROUND

Historically, mastectomies were performed by removing the entire breast, including the breast tissue, breast skin, and nipple-areola complex. More recently, alternative forms of breast surgery have become available. Skin-sparing mastectomy and nipple-sparing/subcutaneous mastectomy with immediate reconstruction and lumpectomy have become surgical procedures of choice because they provide good oncologic surgical results and more favorable aesthetic outcomes. In skin-sparing mastectomy, for example, the submammary fold and breast contour are preserved and skin differences are avoided. In subcutaneous mastectomy, which is viable for patients with cancer free nipple tissue, the nipple-areola complex is preserved.

Such surgical procedures, however, typically have small access incision through which the surgeon accesses the surgical procedure operating region. In subcutaneous mastectomy, for example, an access incision is made around the areola through which the surgeon detaches and removes the breast tissue and gains access to the axilla for staging.

One of the main challenges of using a small access incision is the limited visibility within the surgical procedure operating region. Poor or inadequate visualization into the surgical procedure operating region prolongs surgical procedures and puts the patient and the surgical team at risk (e.g. of burns and cuts). Traditional operating room lighting options are inadequate. Anchored overhead lights, surgery lights on mobile stands, and headlight systems have poor shadow dilution. They create shadows in the surgical procedure operating region caused by the intervening surgical team's bodies or body parts or intervening surgical equipment. Such operating room lighting options, in turn, have to be constantly readjusted to reduce the amount of shadows they cause, delaying the surgery, which increases the risk of infection and morbidity. Moreover, constant readjustment of headlights causes neck and shoulder strain and headaches for the surgeon.

Accordingly, it would be an advantage to provide a surgical tool that overcomes the disadvantages of previous technology.

### SUMMARY

In certain embodiments, an article of manufacture includes a hand-held electrosurgical illumination pencil, which has an elongated body and an illumination source. The elongated body has a proximal and a distal end. The illumination source is coupled to the elongated body at the proximal end. The illumination source is configured: to receive energy from a first energy source, and to project a light field for illuminating a surgical procedure operating region.

In certain embodiments, a hand-held surgical illumination tool includes an elongated body with at least a first end, a cautery electrode, and an illumination source. The cautery electrode extends from the first end of the body and is configured to receive energy from a first energy source. The illumination source is coupled to the body and is configured to: receive energy from a second energy source and project a light field that contains at least a portion of the cautery electrode.

In certain embodiments, a method for illuminating a surgical procedure operating region includes inserting one or more surgical devices into respective slots of a hand-held surgical illumination tool that includes an illumination source. The method for illuminating the surgical procedure operating region further includes using the hand-held surgical illumination tool to perform surgery on a patient within a surgical procedure operating region illuminated by the illumination source.

### BRIEF DESCRIPTION OF THE DRAWINGS

Various embodiments will be better understood from a reading of the following detailed description taken in conjunction with the drawings in which like reference designators are used to designate like elements, and in which:

FIG. 1A is a schematic illustrating a depth of illumination for an illumination source;

FIG. 1B illustrates a bell curve representing a depth of illumination of an illumination source;

FIG. 2A is a schematic of a mammoplasty retractor with an illumination source;

FIG. 2B is a schematic illustrating misdirection of light from a mammoplasty retractor;

FIG. 3A is a schematic illustrating a hand-held surgical illumination tool having an illumination source and a cautery electrode;

FIG. 3B is a schematic of a cross-section of the hand-held surgical illumination tool of FIG. 3A;

FIG. 4A is a schematic illustrating a wireless hand-held surgical illumination tool having an illumination source;

FIG. 4B is a schematic of a cross-section of the hand-held surgical illumination tool of FIG. 4A;

FIG. 5A is a schematic illustrating a hand-held surgical illumination tool having an illumination source and a vacuum source;

FIG. 5B is a schematic of a cross-section of the hand-held surgical illumination tool of FIG. 5A;

FIG. 6 is a schematic illustrating examples of energy supplies for the hand-held surgical illumination tool;

FIG. 7 is a flow chart illustrating a method for performing breast surgery using the hand-held surgical illumination tool;

FIG. 8A is a schematic illustrating a top view of a hand-held surgical illumination tool;

FIG. 8B is a schematic illustrating a front view of the hand-held surgical illumination tool of FIG. 8A;

FIG. 8C is a schematic illustrating a left-side view of the hand-held surgical illumination tool of FIG. 8A;

FIG. 9A is a schematic illustrating a top view of a hand-held surgical illumination tool;

FIG. 9B is a schematic illustrating a front view of the hand-held surgical illumination tool of FIG. 8A;

FIG. 9C is a schematic illustrating a left-side view of the hand-held surgical illumination tool of FIG. 8A;

FIG. 10 is a circuit diagram of an ultraviolet LED lighting for the hand-held surgical illumination tool;

FIG. 11A is a front view of an embodiment of a surgical illumination tool;

FIG. 11B is a perspective view of the surgical illumination tool of FIG. 11A; and

FIG. 12 is an exploded view of the surgical illumination tool of FIG. 11A showing the housing, the light, the battery, and the switch.

### DETAILED DESCRIPTION

A hand-held surgical illumination tool includes an illumination source that is configured to projects light into a surgical procedure operating region, such as a location in which a surgical device is interacting or will interact with a tissue of a patient (e.g., human or animal). In certain embodiments, at least one frequency of the light is within the visible spectrum while in other embodiments at least one frequency of the light is longer or shorter than the visible spectrum. In certain embodiments, the frequency of light is set to produce minimum reflection off the tissue.

In certain embodiments, the hand-held surgical illumination tool has an elongated body of any suitable shape such as substantially cylindrical, rectangular, or conical shape. For example, the hand-held surgical illumination tool has an elongated body like a pencil (e.g., “the hand-held surgical illumination pencil”). Here, the surgeon holds the hand-held surgical illumination tool like a pencil and is able to direct the proximal end of the hand-held surgical illumination tool towards the surgical procedure operating region via hand or arm movements.

In certain embodiments, the hand-held surgical illumination tool includes one or more surgical devices. To illustrate, the one or more surgical devices includes, but is not limited to, one or more of the following: a bone chisel, a cannula, a curette, a cautery electrode, a dilator, a surgical Pinzette, a forcep, a hook, a scalpel, a mammotome, an osteotome, a surgical elevator, a probe, a scissor, a speculum, a surgical spoon, a stapler, a clamp, a trocar, a vacuum source, a laparoscope or a combination thereof.

The surgical device is made of any suitable material, such as plastic, glass, wood, or metal. For example, in some embodiments, the surgical device is made at least in part of transparent, translucent or semitranslucent material such as translucent plastic or glass. In certain embodiments, the surgical device is transparent, translucent or semitranslucent and conductive. When the surgical device is made at least in part of translucent or semitranslucent material, it reduces the chance of the surgical device producing a shadow in the light field of an illumination source that illuminates the surgical procedure operating region. For example, the surgical device that is a cautery electrode is made from one or more organic or inorganic layers of transparent conducting film. To illustrate, the cautery electrode is made of a center portion that is nonconductive translucent plastic (e.g., cast acrylic) that is coated at least in part with conductive translucent plastic such as transparent conducting film including indium tin oxide, fluorine doped tin oxide, and doped zinc oxide.

In certain embodiments, a portion of one or more surgical devices is housed within the body of the hand-held surgical illumination tool or pencil, which can then be actuated to extend out of, or retract into, the body at the proximal end. For example, the surgical device is a scalpel. A portion of the scalpel is housed within the body of the hand-held surgical illumination tool that is then manually actuated, via a sliding nob, to extend the scalpel a predetermined length out of the body of the hand-held surgical illumination tool.

In certain embodiments, the one or more surgical devices are interchangeable such that a first surgical device is removed from the hand-held surgical illumination tool and replaced by a different, second surgical device. The body of the hand-held surgical illumination tool has one or more slots at a proximal end of the hand-held surgical illumination tool. The slot provides sufficient resiliency to receive a first end of the surgical device and provide a stable mechanical and electrical connection with the surgical device.

When the hand-held surgical illumination tool includes a plurality of surgical devices, the surgical procedure operating region is less cluttered than in conventional means that use separate receptacles for the respective surgical devices. For example, when separate surgical devices in separate receptacles are used to one of illuminate, vacuum, or cauterize tissue, the separate surgical device receptacles and multiple hand have to be introduced into surgical procedure operating region, cluttering the area and reducing visibility.

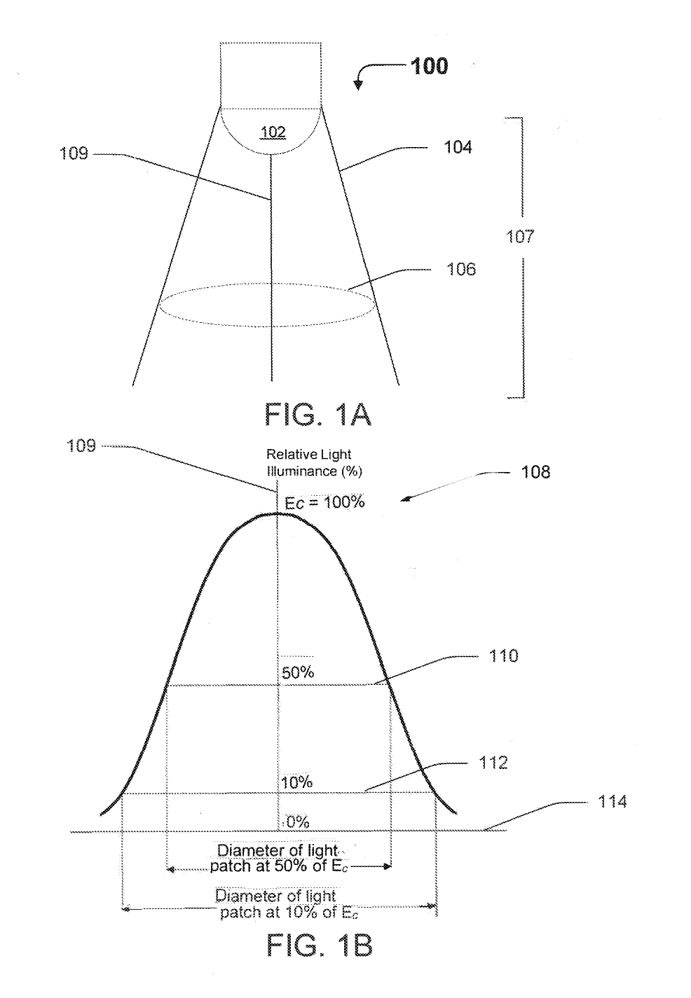

In FIG. 1A a depth of illumination 100 for an illumination source 102 is illustrated. The illumination source 102 is depicted as a convex shape light emitter (e.g., light bulb) that projects light in a substantially conical shaped light field 104. In FIG. 1B, a schematic illustrates a bell curve 108 representing a depth of illumination 107 of the illumination source 102. The center illuminance line 109 is the distance from the illumination source 102 within the light field 104 without any obstruction of the light beam. The depth of illumination 107 is the distance below the emitting surface of the illumination source 102, in which the illuminance reaches about 60% of highest amount of illuminance. The light field diameter 106 is a diameter of a cross section of the light field 104 where the illuminance reaches about 50% of the highest amount of illuminance. In FIG. 1B, the line 110 represents the amount of illuminance at light field diameter 106. The line 112 represents about 10% of the highest amount of illuminance and line 114 represents no illuminance.

The International Electrotechnical Commission (IEC) 60601-2-41—Particular Requirements For The Safety Of Surgical Luminaires And Luminaires For Diagnosis, 2013 is incorporated herein by reference.

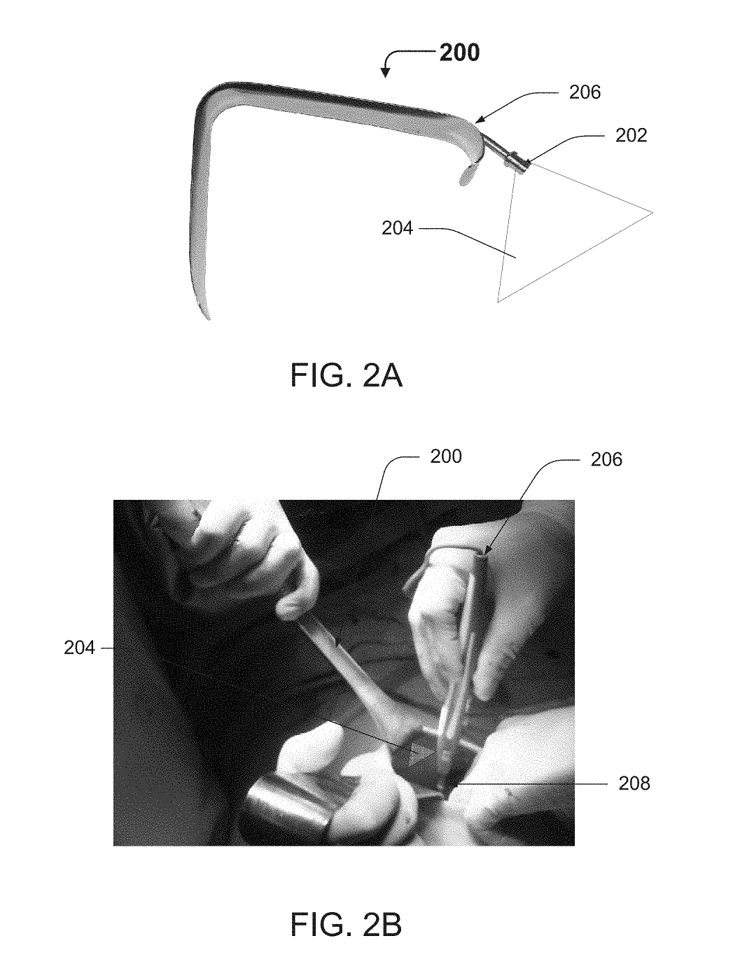

Other illumination sources and light field configurations are also contemplated. The illumination source includes, but is not limited to, any of: a laser light, a fiber optic light, an incandescent light, a light-emitting diode, or halogen light, for example. In certain embodiments, the illumination source has a substantially donut shape emitter in which light emits from substantially a ring. FIG. 2A is a schematic illustrating a mammoplasty retractor 200 that has an illumination source 202 affixed to a dorsal side at a distal end 206 of the retractor 200. The illumination source 202 projects light from the tip of the illumination source 202 that, in turn, produces a light field 204. Typically, a surgeon or an assistant to the surgeon, uses the retractor 200 to pull back a skin of a patient in order to give access to a surgical cavity. However, positioning the retractor 200 during surgery to maintain access to the surgical cavity often does not coincide with a position that illuminates the surgical procedure operating region.

FIG. 2B is a schematic illustrating misdirection of light from the illumination source 202 affixed to the retractor 200. In FIG. 2B a surgeon uses a cauterizer 206 that has a cautery electrode (not shown) at a proximal end 208 of the cauterizer 206. The cautery electrode cuts and/or cauterizes the tissue it comes into contact with during the procedure. Here, the light field 204 from the retractor 200 does not reach the surgical procedure operating region, which is at the edge of the cautery electrode. Even if the surgeon readjusted the retractor 200 to direct the light field 204 towards the cauterizer 206, the cauterizer 206 body produces an intervening shadow that obstructs the light from reaching the surgical procedure operating region. Consequently, the surgeon has inadequate visualization of the tissue planes and anatomical landmarks within the surgical procedure operating region and lower control of the excisional tissue.

The illumination source 202 affixed to the retractor 200 also poses a burn and fire hazard. The illumination source 202 produces heat. As the surgeon or surgeon's assistant reaches into the surgical cavity, she is exposed to potentially burning her hand. Moreover, surgical cloth is often stuffed into the surgical cavity to absorb blood and other bodily fluids. Exposure of the surgical cloth to the illumination source 202 poses a fire hazard.

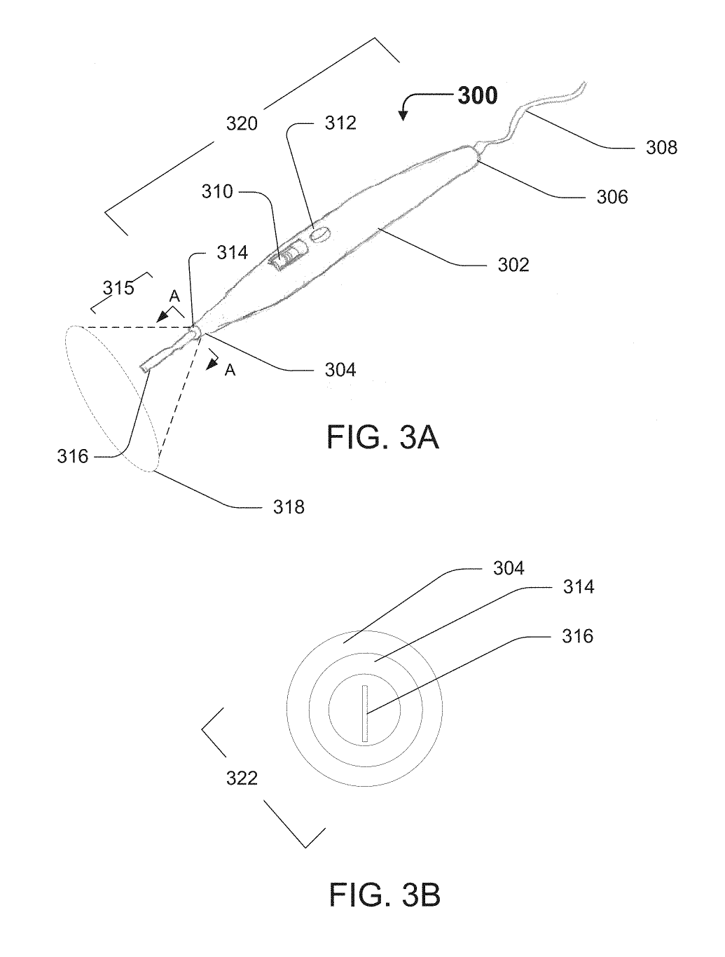

Referring to FIGS. 3A and 3B, a hand-held electro surgical illumination tool 300 has an elongated body 302, illustrated as substantially cylindrical, with a proximal end 304 and a distal end 306. FIG. 3B is a cross-section of the hand-held surgical illumination tool 300 at “A.” In certain embodiments, a distance 320 between the proximal end 304 and the distal end 306 is between 5 cm to 30 cm, more specifically between 10 cm to 20 cm, and preferably between 13 cm to 18 cm and a cross-sectional diameter 322 at the proximal end 304 is between 3 mm to 300 mm, more specifically between 5 mm to 150 mm, and preferably between 10 cm to 20 cm. In certain embodiments, the exterior of the hand-held surgical illumination tool 300 has indentations for the placement of fingers or is textured for better gripping.

The body 302 of the hand-held surgical illumination tool 300 is made of any suitable material, such as a polymer, plastic, a metal, wood, glass, transparent or semitransparent material, or a combination thereof. In certain embodiments the hand-held surgical illumination tool 300 is for one-time use, such as being disposable. A wire 308 at the distal end 306 electrically couples the hand-held surgical illumination tool 300 to an external energy source (not shown). When powered by electrical means, the hand-held surgical illumination tool 300 is also referred to as a hand-held electrosurgical illumination tool.

In the embodiment shown in FIG. 3A a cautery electrode 316 extends from the proximal end 304 of the hand-held surgical illumination tool 300. The cautery electrode 316 is made from, or is coated with, a conductive material such as stainless steel. In certain embodiments, the cautery electrode 316 is disposable and/or replaceable. Although illustrated as an electrode with a beveled tip, the cautery electrode 316 can have any functional form. For example, in certain embodiments, the cautery electrode 316 is a needle, a blade, a wand, or a combination thereof.

A length 315 of the cautery electrode 316 is within a range of about 1 cm to about 30 cm. In certain embodiments, a surgeon uses a plurality of interchangeable cautery electrodes 316 of different lengths based on characteristics of the surgical procedure operating region. To illustrate, the surgeon initially uses the hand-held surgical illumination tool 300 with the cautery electrode 316 that is 5 cm in length to cut the epidural skin of a patient. The surgeon then replaces the 5 cm cautery electrode 316 during surgery to a 10 cm cautery electrode 316 to reach tissue deep within an armpit of the patient. Alternatively, in certain embodiments, a length of the cautery electrode 316 is variable without interchanging the cautery electrode 316. For example, a portion of the cautery electrode 316 is housed within the body of the hand-held surgical illumination tool 300, which can then be actuated to extend out of, or retract into, the body 302 at the proximal end 304.

An illumination source 314 is coupled to the body 302 of the hand-held surgical illumination tool 300 at the proximal end 304. Although one illumination source 314 is illustrated, in some embodiments, the hand-held surgical illumination tool 300 has more than one illumination source. The illumination source 314 is shaped substantially like a donut and is coupled to a proximal end of the cautery electrode 314. The illumination source 314 is configured to produces a light field 318 into the surgical procedure operating region, which includes at least a portion of the cautery electrode 316 used for cautery and/or excision.

One or more actuation controls (such as knobs; buttons; slide switches, or foot pedals) electrically control at least one of the cautery electrode 316 and the illumination source 314 by opening or closing switches to one or more energy sources. In the embodiment of FIG. 3A, a slide switch 310 coupled to the body 302 controls the output of the cautery electrode 316 between off, cut, cauterize, and both cut and cauterize by opening or closing respective switches connecting the cautery electrode 316 to an energy source. The button 312 controls the light source 314 to turn on or off.

As previously described, the illumination source 314 is any suitable source of light for surgical procedures. For example, the illumination source 314 is a laser light, a halogen light, light from an optical fiber, or a combination thereof. In certain embodiments, the light from the illumination source produces one or more frequencies selected from the group consisting of: a frequency within a humanly visible spectrum, a frequency shorter than in the humanly visible spectrum; and a frequency longer than in the humanly visible spectrum. For example, the illumination source 314 produces a blue light or a red light, or a white light that includes all the frequencies in the visible spectrum.

In certain embodiments, the illumination source 314 acts as an antimicrobial. To illustrate, the illumination source 314 projects a narrow spectrum of Ultra-Violet (UV) light—between 200 nm to 300 nm, preferably about 207 nm wavelength, for example. At this wavelength range, the light destroys bacteria while leaving human tissue unaffected. At this wavelength range the UV light is smaller in size than a nucleus of human cells, therefore not damaging them. However, because bacteria are smaller than human cells, the UV light reaches the bacteria and kills them.

In certain embodiments, the illumination source 314 projects light for in-vivo imaging during surgery. For example, illumination source 314 projects light with near-infrared radiation (600 to 1,200 nm wavelengths, modulated in the megahertz-to-gigahertz range) to produce diagnostic images that are rendered on a monitor, or other rendering means.

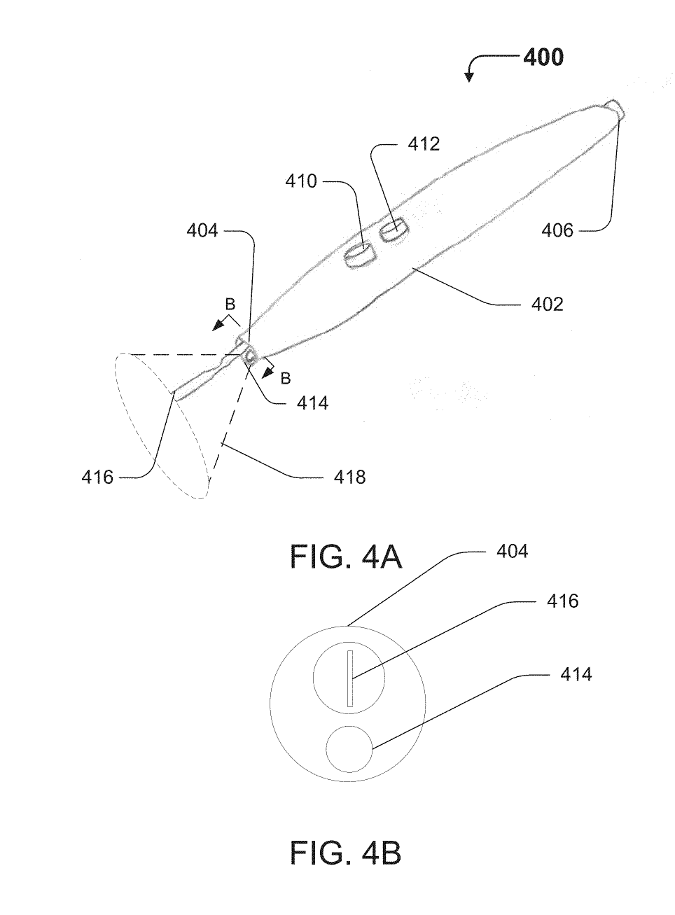

Other configurations for the hand-held surgical illumination tool are also contemplated. For example, the illumination source at the proximal end of the hand-held surgical illumination tool is situated next to the cautery electrode rather than around the cautery electrode. Examples are provided in FIGS. 4A through 6B.

Referring to FIGS. 4A and 4B, a hand-held surgical illumination tool 400 has an elongated body 402, illustrated as substantially cylindrical, with a tapered proximal end 404 and a distal end 406. The hand-held surgical illumination tool 400 is wireless, powered by an internal battery (not shown). FIG. 4B illustrates a cross-section of the hand-held surgical illumination tool 400 at “B.” A cautery electrode 416 extends from the proximal end 404 of the hand-held surgical illumination tool 400. The illumination source 414 is coupled to the proximal end 404. The illumination source 414 is configured to produce a light field 418 that contains at least a portion of the cautery electrode 414, illuminating the surgical procedure operating region when the cautery electrode 416 is used. Coupled to the body 402 are one or more actuation controls, for electrically controlling at least one of the cautery electrode 416 and the illumination source 414 by opening or closing switches to one or more energy sources. In the embodiment of FIG. 4A, a button 410 controls the on/off of the cautery electrode 416 and the button 412 controls the on/off of the illumination source 414.

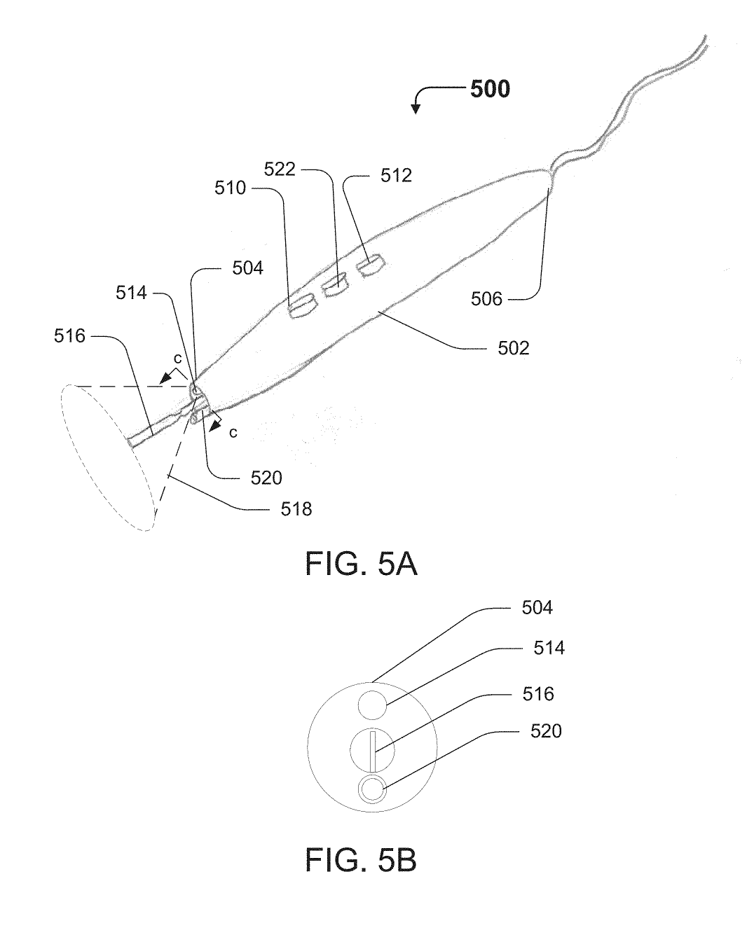

Referring to FIGS. 5A and 5B, a hand-held surgical illumination tool 500 has an elongated body 502, illustrated as primarily cylindrical, with a proximal end 504 and a distal end 506. FIG. 5B illustrates a cross-section of the hand-held surgical illumination tool 500 at “C.” A cautery electrode 516 extends from the proximal end 504 of the hand-held surgical illumination tool 500. A vacuum source 520 is coupled to the proximal end 504 of the hand-held surgical illumination tool 500 and configured to produce suction at the proximal end 504. In certain embodiments, the vacuum source 520 includes a tubing housed within the hand-held surgical illumination tool 500 and connected to a suctioning means at the distal end 506.

The illumination source 514 is coupled to the proximal end 504. The illumination source 514 produces a light field 518 that contains at least a portion one or more of: the cautery electrode 516 and the vacuum source 520. Coupled to the body 502 are one or more actuation controls, for electrically controlling at least one of: the cautery electrode 516, the illumination source 514, and the vacuum source 520 by opening or closing respective switches to one or more energy sources. In the embodiment of FIG. 5A, a button 510 controls the on/off of the cautery electrode 516, the button 512 controls the on/off of the light source 514, and the button 522 controls the on/off of the vacuum source 520.

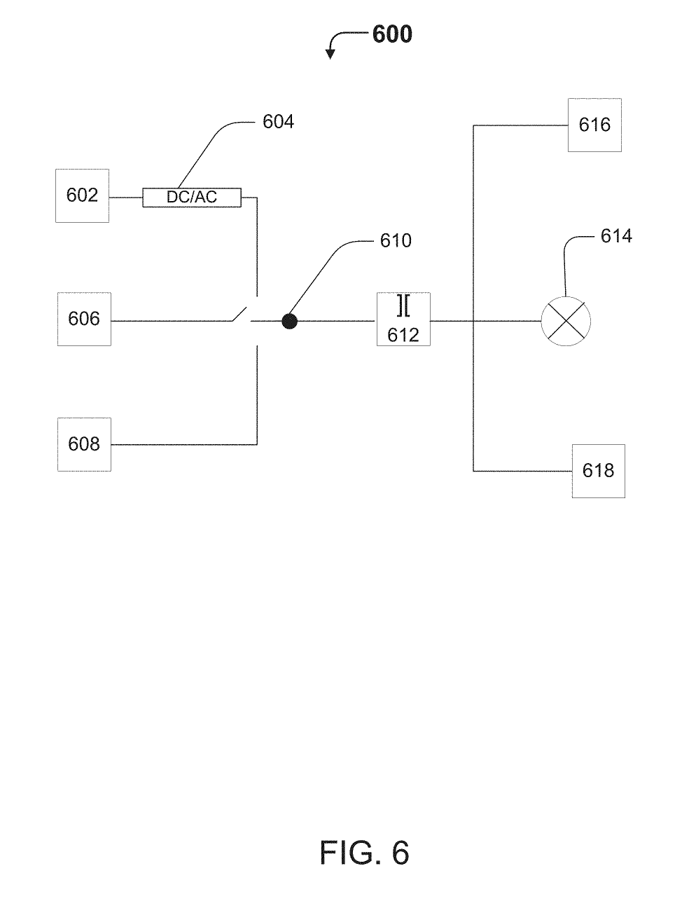

Referring to FIG. 6, a schematic illustrates an exemplary electrical circuit 600 for the hand-held surgical illumination tool (e.g., the hand-held surgical illumination tools 300, 400 and 500). The electrical circuit 600 includes one or more energy sources. In FIG. 6, three energy sources are depicted: a battery 602, a generator 606, and a photovoltaic fuel cell 608. In other embodiments more or less energy sources are used, such as a programmable power supply or a combination thereof. A direct current to alternative current converter 604 is electrically coupled to the battery 602. The one or more energy sources are electrically coupled to a switch 610 that is, in turn, electrically coupled to a transformer 612. The transformer 612 is electrically coupled to the illumination source 614. In certain embodiments, transformer 612 is further electrically coupled to one or more surgical devices, represented in FIG. 6 as surgical device 616 and 618 (e.g., a cautery electrode and a vacuum source, respectively).

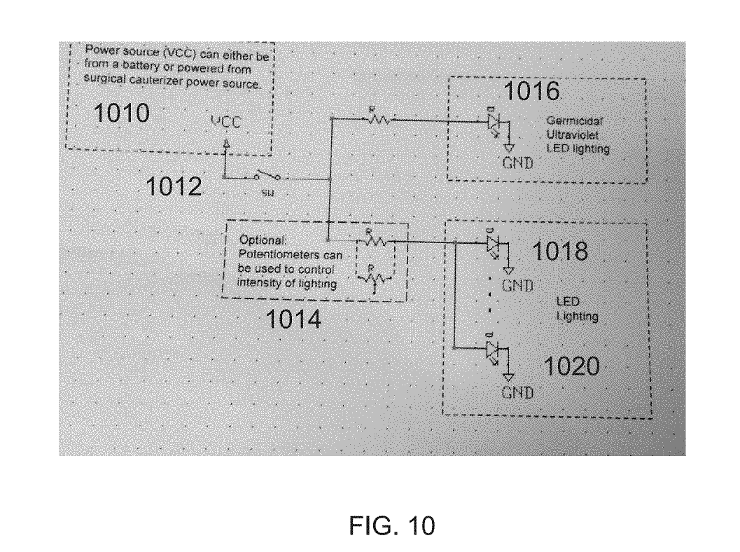

Referring to FIG. 10, an exemplary circuit diagram of an ultraviolet LED lighting for the hand-held surgical illumination tool is illustrated. A power source 1010, such as a battery or a power source of a surgical cauterizer, is electrically coupled to a switch 1012 that, in turn, is coupled in parallel to each of: a potentiometer 1014 used to control an intensity of a first set of lighting and to a germicidal ultraviolet LED lighting 1016 and ground. The potentiometer 1014 is electrically connected to a second set of LED lighting 1018 and 1020 (e.g., ultraviolet and/or non-ultraviolet lighting) and ground.

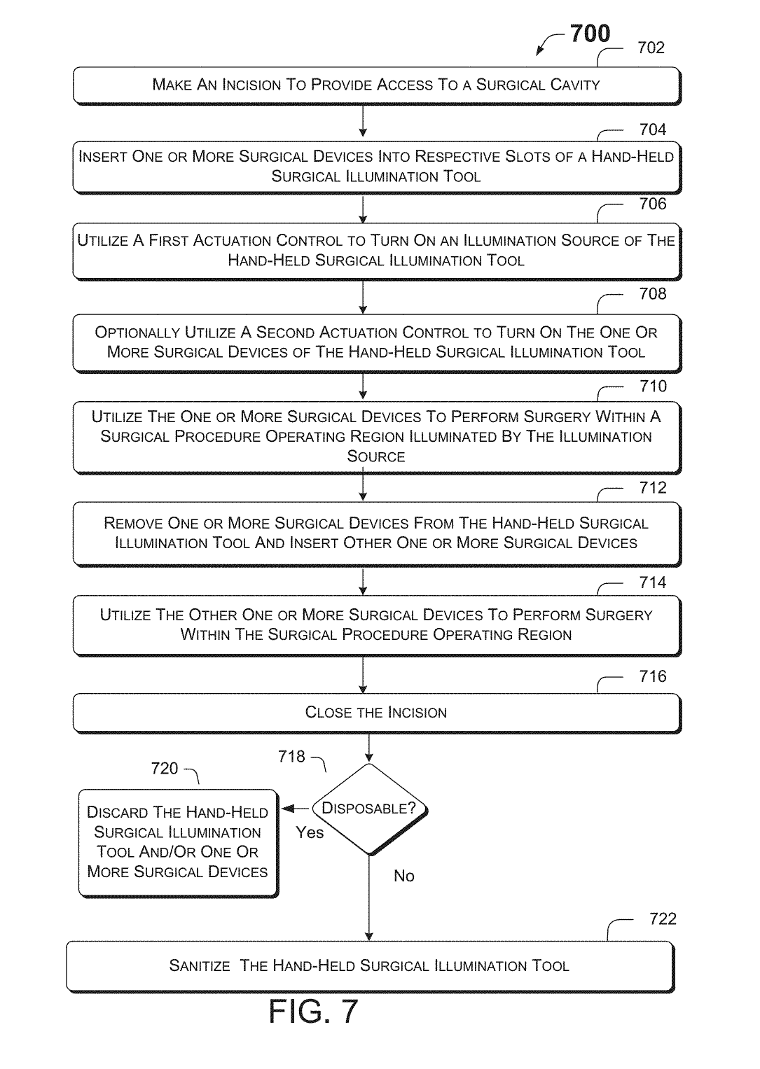

Referring to FIG. 7, a method 700 provides steps for utilizing the hand-held surgical illumination tool. At step 702, an incision is made to provide access to a surgical cavity with a patient. At step 704, one or more surgical devices are inserted into respective slots of a hand-held surgical illumination tool having an illumination source. In certain embodiments, one or more surgical devices are removably inserted into respective slots of a hand-held surgical illumination tool such that the one or more surgical devices can be removed from the hand-held surgical illumination tool at a subsequent time. At step 706, a first actuation control is utilized to turn on the illumination source of the hand-held surgical illumination tool. At step 708, a second actuation control is utilized to turn on the one or more surgical devices of the hand-held surgical illumination tool. At step 710, the one or more surgical devices of the hand-held surgical illumination tool is utilized to perform surgery within a surgical procedure operating region illuminated by the illumination source. At step 712, the one or more surgical devices are removed from the hand-held surgical illumination tool and replaced with other one or more surgical devices. At step 714, the other one or more surgical devices of the hand-held surgical illumination tool is utilized to further perform surgery within the surgical procedure operating region illuminated by the illumination source. At step 716, the incision is closed. At step 718, if the hand-held surgical illumination tool and/or one or more surgical device are disposable, the method moves to step 720 in which the hand-held surgical illumination tool is discarded. If the hand-held surgical illumination tool is not disposable at step 718, the method moves to step 722 in which the hand-held surgical illumination tool is sanitized for further use.

For example, in a nipple-sparing mastectomy (NSM), a surgeon makes a peri areolar incision with lateral extension in the patient (step 702). The surgeon, surgeon's assistant, or other health care provider removably inserts a cautery electrode into a respective slot of a hand-held surgical illumination tool having an illumination source (step 704). In certain embodiments, the cautery electrode is permanently coupled to the hand-held surgical illumination tool during manufacturing and step 704 is omitted. The surgeon, surgeon's assistant, or other health care provider pushes a first button to turn on the illumination source and a second button to turn on the cautery electrode (steps 706 and 708, respectively). The surgeon utilizes the hand-held surgical illumination tool to remove the breast tissue and cauterize the respective remaining tissue within the surgical procedure operating region illuminated by the illumination source (step 710). The cautery electrode is removed from the hand-held surgical illumination tool and a portion of forceps is inserted into a respective slot on the hand-held surgical illumination tool (712). The surgeon utilizes the forceps to further perform surgery (714) by using the forceps to suture the incision (716). The cautery electrode (720) and the hand-held surgical illumination tool are each discarded while the forceps are sanitized (722).

In certain embodiments, individual steps recited in various processes are combined, eliminated, or reordered. The schematic flow chart diagram included is generally set forth as a logical flow-chart diagrams (e.g., FIG. 7). As such, the depicted order and labeled steps are indicative of one or more embodiment of the presented method. In certain embodiments, other steps and methods are conceived that are equivalent in function, logic, or effect to one or more steps, or portions thereof, of the illustrated method. Additionally, the format and symbols employed are provided to explain the logical steps of the method and are understood not to limit the scope of the method. Although various arrow types and line types are employed in the flow-chart diagrams, they are understood not to limit the scope of the corresponding method. Indeed, some arrows or other connectors may be used to indicate only the logical flow of the method. For instance, an arrow indicates a waiting or monitoring period of unspecified duration between enumerated steps of the depicted method. Additionally, the order in which a particular method occurs may or may not strictly adhere to the order of the corresponding steps shown.

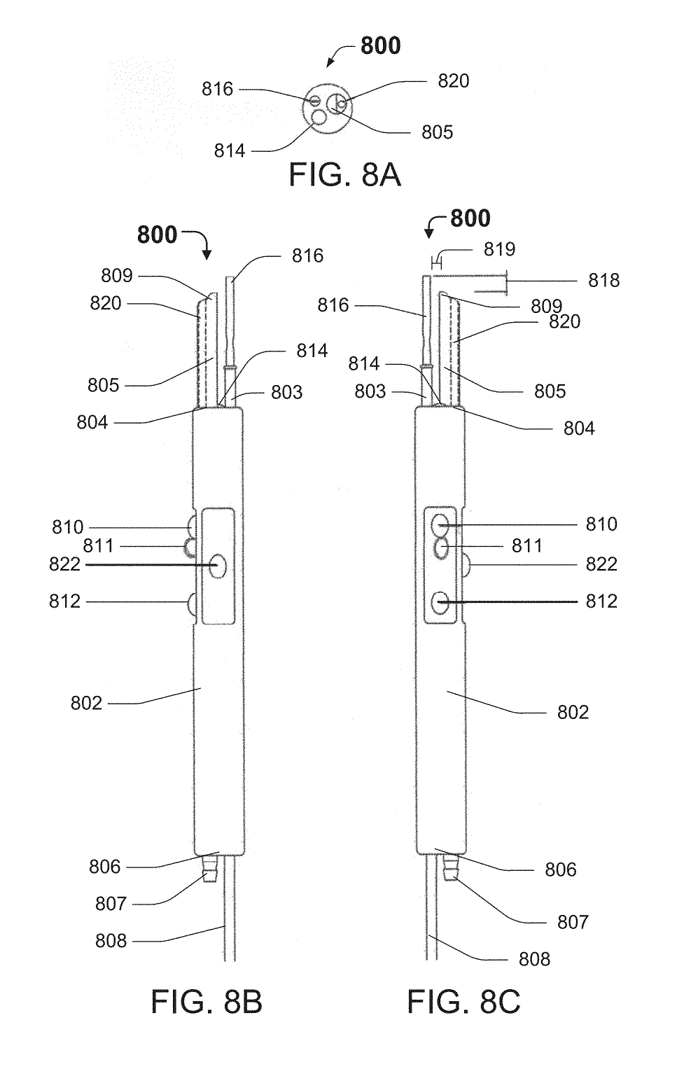

Referring to FIGS. 8A-8C, a hand-held surgical illumination tool 800 has an elongated body 802, illustrated as primarily cylindrical, with a proximal end 804 and a distal end 806. FIG. 8A illustrates a top view of the hand-held surgical illumination tool 800 while FIG. 8B illustrates a front view and FIG. 8C illustrates a left-side view of the hand-held surgical illumination tool 800. A cautery electrode 816 is operatively supported by a collar 803 that extends from the proximal end 804 of the hand-held surgical illumination tool 800. An illumination source 814 is coupled to the proximal end 804. The illumination source 814 produces a light field that contains at least a portion of one or more of: the cautery electrode 816 and a vacuum source 820. The vacuum source 820 is coupled to the proximal end 804 of the hand-held surgical illumination tool 800 and configured to produce suction. In certain embodiments, the vacuum source 820 includes a tubing housed within the hand-held surgical illumination tool 800 and connected to a suctioning means 807 at the distal end 806.

A probe 805 is coupled to the proximal end 804 of the hand-held surgical illumination tool 800. In certain embodiments, the vacuum source 820 and the probe 805 independently extend from the proximal end 804; alternatively or in combination, in certain embodiments, the vacuum source 820 and the probe 805 are mutually housed in a structure 809 that extends from the proximal end 804. In certain embodiments, a difference in height 818 extending out of the proximal end 804 between the cautery electrode 816 and the probe 805 is between about 0.1 cm to about 0.8 cm and the medial-lateral distance 819 between cautery electrode 816 and the probe 805 is about 0.1 cm to about 0.8 cm. During use, the probe 805 exerts a force on the tissue ahead of the cautery electrode 816, producing tension in the tissue prior to cauterization. For example, in FIG. 8C, if the hand-held surgical illumination tool 800 moves towards the right, the probe 805 pushes the tissue to produce a tension in the tissue volume within the medial-lateral distance 819. The tension stretches the tissue ahead of the cautery electrode 816 compressing the corresponding blood vessels and fat within the tissue. Stretching the tissue in advance of the cautery electrode 816 provides more control over treatment of the tissue by facilitating exposure to the tissue; and reduces bleeding, charring, desiccation, and sticking of the tissue to the cautery electrode 816. This, in turn, causes less damage to the tissue and reduces the probability of bacterial infection.

Electrical impedance of tissue is a measure of the tissue's opposition to the flow of alternating electric current at various frequencies. Cutting and/or cauterizing a relaxed tissue at about 2K ohms impedance usually cuts and/or cauterizes at current intensities ranging from about 60 mA to about 240 mA for minimal to aggressive cutting/dissecting/hemostatic effects, respectively. A tissue in tension has a different impedance than tissue that is relaxed. The probe 805 produces tension in the tissue such that it changes the impedance of the tissue. The amount of current necessary to cut/dissect/cauterize the tissue also changes. In certain embodiments, use of the probe 805 reduces the amount of power necessary to cut/dissect/cauterize the tissue.

Coupled to the body 802 are one or more actuation controls, for electrically controlling at least one of: the cautery electrode 816, the illumination source 814, and the vacuum source 820 by opening or closing respective switches to one or more energy sources coupled to the hand-held surgical illumination tool 800 by wire 808. A button 810 controls the on/off of the cautery electrode 816, the button 812 controls the on/off of the light source 814, the button 822 controls the on/off of the vacuum source 820, and the button 811 simultaneously controls the on/off of the cautery electrode 816 and the vacuum source 820. In certain embodiments, the hand-held surgical illumination tool 800 has a power actuation control (e.g., a knob for manual control; not shown) to change an amount of power (e.g., current and/or voltage and/or corresponding wave form) delivered to the tissue by the hand-held surgical illumination tool 800.

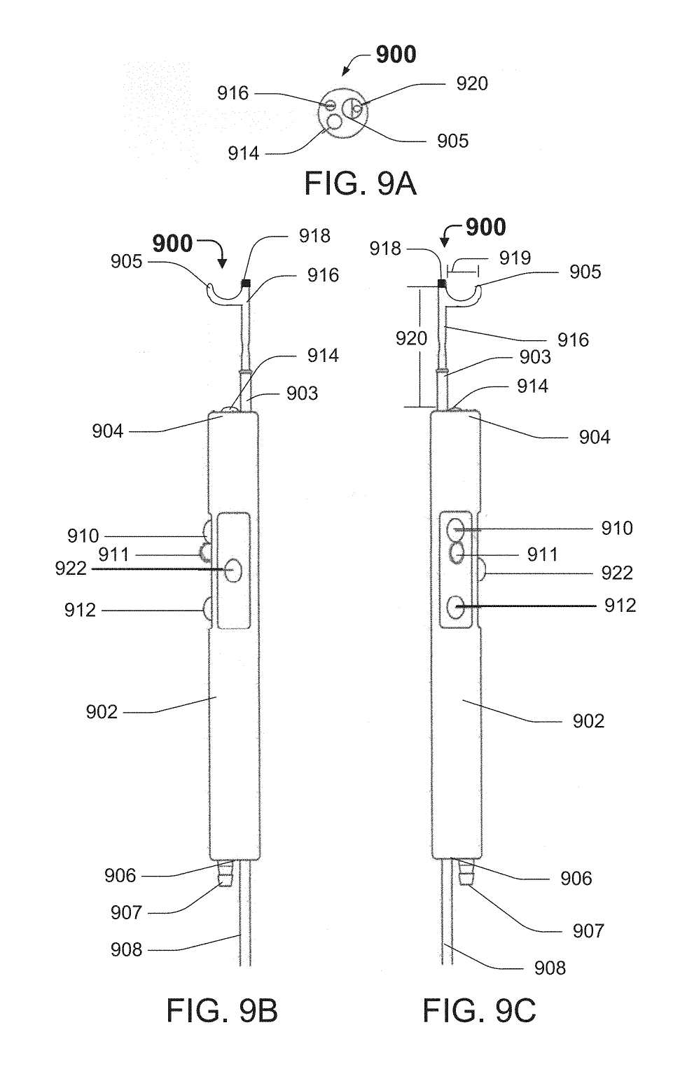

Referring to FIGS. 9A-9C, a hand-held surgical illumination tool 900 has an elongated body 902, illustrated as primarily cylindrical, with a proximal end 904 and a distal end 906. FIG. 9A illustrates a top view of the hand-held surgical illumination tool 900 while FIG. 9B illustrates a front view and FIG. 9C illustrates a left-side view of the hand-held surgical illumination tool 900. An illumination source 914 is coupled to the proximal end 904. The illumination source 914 produces a light field that contains at least a portion of the cautery electrode 916.

A cautery electrode 916 is optionally supported by a collar 903 that extends from the proximal end 904 of the hand-held surgical illumination tool 900. In some embodiments this collar 903 is not present. In certain embodiments, the cautery electrode 916 is at least in portion translucent or semitranslucent; for example, the cautery electrode 916 is made of at least in part of a plastic or glass that allows light to pass through the translucent part of the cautery electrode 916. When the cautery electrode 916 is made at least in part of translucent or semitranslucent material, it reduces the chance of the cautery electrode 916 producing a shadow in the light field.

In FIGS. 9B and 9C, a body 920 of the cautery electrode 916 is made of a translucent material while a tip 918 is made of a material that allows for electrical conduction, such as metal. For illustrative purposes only, the longitudinal length of the tip 918 is between 0.1 cm to 5 cm. In certain embodiments, the energy source is electrically coupled to the tip 918 via wires that are housed within cylindrical canals within the body 920 of the cautery electrode 916 (not shown). Alternatively, or in combination, the energy source is electrically coupled to the tip 918 via a means that does not require direct wiring, such as through a capacitive means.

In FIGS. 9A-9C, a probe 905 is coupled and/or is part of the cautery electrode 916. Alternatively, or in combination, the probe 905 and cautery electrode 916 are mutually housed in a structure that extends from the proximal end 904. In certain embodiments, the medial-lateral distance 919 between cautery electrode 916 and the probe 905 is about 0.01 cm to about 2 cm. During use, the probe 905 exerts a force on the tissue ahead of the cautery electrode 916, producing tension in the tissue prior to cauterization. For example, in FIG. 9C, if the hand-held surgical illumination tool 900 moves towards the right, the probe 905 pushes the tissue to produce a tension in the tissue volume within the medial-lateral distance 919. The tension stretches the tissue ahead of the tip 918 compressing the corresponding blood vessels and fat within the tissue.

Coupled to the body 902 are one or more actuation controls, for electrically controlling at least one of: the cautery electrode 916 and the illumination source 914 by opening or closing respective switches to one or more energy sources coupled to the hand-held surgical illumination tool 900 by wire 908. A button 910 controls the on/off of the cautery electrode 916, the button 912 controls the on/off of the light source 914, the button 922 controls the actuation up and down of the cautery electrode 916, and the button 911 simultaneously controls the on/off of the cautery electrode 916 and the light source 914. In certain embodiments, the hand-held surgical illumination tool 900 has a power actuation control (e.g., a knob for manual control; not shown) to change an amount of power (e.g., current and/or voltage and/or corresponding wave form) delivered to the tissue by the hand-held surgical illumination tool 900.

Referring to FIGS. 11A, 11B, and 12 a surgical illumination tool 1110 is shown. FIG. 12 is an exploded view of the illumination tool 1110 showing a housing 1112, a switch 1114, a light source 1116, a ring 1118, and an energy source 11120, such as battery or power source. In accordance with some embodiments of the invention, the housing 1112 includes a top portion 1112a and a bottom portion 1112b. In accordance with some embodiments of the invention, the housing 1112 may be one piece (not shown). The housing 1112 holds the battery or power source 1120 and the light source 1116. The light source 1116 can be any type of light source, including an LED and power to the light source 1116 is controlled by a switch 1114. The ring 1118 couples to or engages a surgical instrument (not shown in FIG. 12), such as the hand-held surgical instrument discussed above. The internal portion of the ring 1118 is designed to couple to or engage the surgical instrument. The shape and design of the internal portion of the ring 1118 varies to matingly engage the surgical instrument. For example, the internal portion may be cylindrical in shape, where the from and back diameters of the opening are the same. In accordance with other embodiments, the diameter of the front portion of the opening and the diameter of the back portion of the opening of the ring 1118 may be different, such that the opening is tapered. Also, as shown in accordance with other embodiments, the ring 1118 includes teeth portions 1118a that matingly match up to a specific design of a surgical instrument. The number of teeth 1118a and the shape of each teeth 1118a vary depending on the shape of the surgical instrument that the ring 1118 will engage. In accordance with some embodiments of the invention, the teeth 1118a are identical in shape and size. In accordance with some embodiments of the invention, the teeth 1118 vary is shape and/or size.

While various embodiments have been described above, it should be understood that they have been presented by way of example only, not limitation, and various changes in form and details may be made. Any portion of the apparatus and/or methods described herein may be combined in any combination, except mutually exclusive combinations. The embodiments described herein can include various combinations and/or sub-combinations of the functions, components and/or features of the different embodiments described.

The described features, structures, or characteristics of the invention may be combined in any suitable manner in one or more embodiments. In the above description, numerous specific details are recited to provide a thorough understanding of embodiments of the invention. One skilled in the relevant art will recognize, however, that the invention may be practiced without one or more of the specific details, or with other methods, components, materials, and so forth. In other instances, well-known structures, materials, or operations are not shown or described in detail to avoid obscuring aspects of the invention.

Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” “in certain embodiments,” and similar language throughout this specification may, but do not necessarily, all refer to the same or different embodiment. It is noted that, as used in this description, the singular forms “a,” “an” and “the” include plural referents unless the context clearly dictates otherwise.

What is claimed is:

1. A hand-held electrosurgical illumination pencil comprising:
an elongated body having a proximal end and a distal end; and
an illumination source coupled to the elongated body at the proximal end, wherein illumination source is configured to:
project light into a light field for illuminating a surgical procedure operating region; and
receive energy from a first energy source.

2. The hand-held electrosurgical illumination pencil of claim 1, further comprising a cautery electrode extending from the proximal end of the body, wherein the cautery electrode is configured to receive energy from a second energy source.

3. The hand-held electrosurgical illumination pencil of claim 2, further comprising an actuation control coupled to the body that one of opens and closes: a first switch connecting the cautery electrode to the first energy source; and a second switch connecting the illumination source to the second energy source.

4. The hand-held electrosurgical illumination pencil of claim 2, wherein the first energy source is the same as the second energy source.

5. The hand-held electrosurgical illumination pencil of claim 2, wherein at least one of the first energy source and the second energy source is selected from the group consisting of: a battery, a generator, a photovoltaic fuel cell, a programmable power supply, or a combination thereof.

6. The hand-held electrosurgical illumination pencil of claim 1, further comprising a vacuum source configured to produce suction at the proximal end.

7. The hand-held electrosurgical illumination pencil of claim 1, wherein the body is substantially cylindrical.

8. The hand-held electrosurgical illumination pencil of claim 1, wherein the light has one or more frequencies within a humanly visible spectrum.

9. The hand-held electrosurgical illumination pencil of claim 1, wherein the light has one or more frequencies selected from the group consisting of: a first frequency shorter than in a humanly visible spectrum; and a second frequency longer than in the humanly visible spectrum.

10. The hand-held electrosurgical illumination pencil of claim 1, further comprising a probe extending from the proximal end of the body.

11. The hand-held electrosurgical illumination pencil of claim 10, wherein the probe is made at least in part of translucent material.

12. A hand-held surgical illumination tool comprising:
an elongated body with at least a first end;
a cautery electrode extending from the first end of the body, wherein the cautery electrode is configured to receive energy from a first energy source; and
an illumination source coupled to the body, wherein the illumination source is configured to:
project a light field that contains at least a portion of the cautery electrode; and
receive energy from a second energy source.

13. A method for illuminating a surgical procedure operating region, the method comprising:
inserting one or more surgical devices into respective slots of a hand-held surgical illumination pencil, wherein the hand-held surgical illumination pencil includes an illumination source; and
using the hand-held surgical illumination pencil to perform surgery on a patient within a surgical procedure operating region illuminated by the illumination source.

14. The method of claim 13, further comprising utilizing an actuation control to turn on at least one of: the illumination source and the one or more surgical devices.
